(19) United States
(12) Patent Application Publication
(10) Pub. No.: US 2010/0121300 A1
(43) Pub. Date: May 13, 2010

(54) Nursing Bra Pad

(75) Inventor: Elly Hann, La Jolla, CA (US)

Correspondence Address: PROCOPIO, CORY, HARGREAVES & SAVITCH LLP, 530 B STREET, SUITE 2100, SAN DIEGO, CA, 92101, US

(73) Assignee: PERSIMMON SCIENTIFIC, INC., La Jolla, CA (US)

(21) Appl. No.: 12/618,584

(22) Filed: Nov. 13, 2009

Related U.S. Application Data
(60) Provisional application No. 61/114,067, filed on Nov. 13, 2008.

(51) Int. Cl. A61F 13/15

(52) U.S. Cl. 604/385.07

(57) ABSTRACT

A nursing bra pad has a concave, cup-shape formed from a rounded section of multi-layer composite textile having a first, skin contact layer formed from a non-shearing, moisture vapor permeable woven fabric of synthetic filament yarns. A second layer and optional third layer are formed from moisture absorbing and retaining fabrics. The second layer is a breathable fabric that draws moisture from the inner to the outer surface of the fabric. The optional third microfiber layer provides moisture retention and may be positioned between the first layer and the second layer or between the second and fourth layers. The fourth layer, which may optionally be coated with polyurethane, is formed from a breathable, waterproof fabric woven from synthetic yarn to allow moisture vapor transfer away from the skin. The three or four layers are stitched together along their edges. The layers are stitched to generally conform to the shape of the breast.

### RELATED APPLICATIONS

This application claims the priority of U.S. Provisional Application No. 61/114,067, filed on Nov. 13, 2008, which application is incorporated herein by reference.

### BACKGROUND OF THE INVENTION

While not every nursing mother experiences leaking of breast milk, many do, requiring the use of a nursing bra pad in conjunction with a nursing bra. Nursing bra pads, which are placed against the inside surface of each bra cup, are available in both washable and disposable forms. Disposable pads are typically made of layers of non-woven paper or synthetics with a super-absorbent diaper-like material in the middle layer and often include a plastic lining to prevent moisture from being absorbed into the bra cup. Unless the plastic liner is breathable, it can impede the healing of traumatized nipples or make nipple infections or breast yeast infections more likely. Since the pads need to be changed when they become wet, they may need to be changed frequently, which could become expensive. Furthermore, women who are environmentally conscious may find disposal of large numbers of plastic lined, slowly biodegradable pads to be objectionable.

Washable pads, which are typically made of 100% cotton, such as flannel, or hemp tend to be less expensive in the long run than disposable, however a ready supply of clean, dry pads must be kept at hand for changing. Care must be taken during washing to avoid reduction of the pad's absorbency, and fabric softener is not recommended for this reason. On the other hand, repeated washings without fabric softener could cause the fabric to lose its softness, increasing shear and friction, and reducing comfort. Available washable pads suffer from abrasive surface, lack of breathability, and/or poor ability to conform to the breast shape. Odor control and moisture wicking can be problems as well.

Other commercially available nursing pads include 100% wool pads, which tend to be bulky and relatively expensive, and must be hand washed.

### SUMMARY OF THE INVENTION

The present invention provides an alternative to existing nursing brad pads that avoid many of the drawbacks of existing commercially-available pads. The inventive pad is a multi-layered textile assembly that possesses all of the qualities that are important for providing improved comfort, moisture handling, breathability, shear reducing, rapid drying and anti-microbial, while being durable, launderable and lightweight.

In one aspect of the invention, a bra pad has a cup-shape (concave) formed from a generally rounded shape of a multi-layer composite textile. The rounded shape may be circular, oval (elongated) or a polygon, such as a hexagon, octagon, decagon, etc., that is effectively rounded. The composite textile has a first, skin contact layer formed from a woven fabric of synthetic filament yarns woven to provide air and moisture vapor permeability and a non-shearing surface, with a thickness of less than 1 mm, the skin contact layer having an inner, skin contact surface and a bottom surface; interchangeable second and third layers are formed from moisture absorbing and retaining fabrics. The second layer is formed from a breathable microfiber fabric woven from synthetic fibers having a cross-sectional shape that includes a plurality of voids for generating a capillary effect for moisture absorption from one surface to moisture retention on the opposite surface of the fabric, thus ensuring that the bra and clothing stay dry. The third layer has the additional quality of moisture absorption retention and is preferably the same shape as the first two layers. In one embodiment, the third layer may have a semi-rounded shape, e.g., a half circle, providing additional absorbency in the area where the nipple contacts the lower portion of pad. The third layer may be positioned between the first layer and the second layer or it may be sandwiched between the second layer and the fourth layer. The fourth, outer layer is formed from a breathable, waterproof fabric having a thickness of less that 1 mm that is woven from synthetic yarn such as nylon or polyester to produce a moisture vapor transfer rate within the range of 5,000 to 20,000 grams per meter squared per 24 hours. In a preferred embodiment, the fourth layer is formed from a polyurethane (PU)-coated nylon fabric. A coating of TEFLON® (PTFE) may also be used. The four layers are stitched together along their outer curved perimeters. In one embodiment, the concave shape is formed by creating one or more pleats or darts in the upper portion of the pad to so that the pad generally fits the shape of the breast. The pleats may be formed at the same time the layers are stitched together by folding the fabric as the edges are stitched. Alternatively, the pleats may be formed after the layers are sewn together. In a second, preferred embodiment, pleats are omitted to avoid the presence of seams or stitching in the center pad body and are replaced by a number of smooth puckers that are formed in the first of a two-step sewing process. The first sewing step involves a stitch through all layers near their edges, which causes a slight puckering of the combined layers near the stitches. This creates a gentle curve that will allow the pad to at least partially conform to the inner surface of the user's bra cup without requiring any stitching in the center of the pad that might irritate the wearer's breast.

In an exemplary embodiment, a four-layer textile assembly includes an upper layer, typically for direct skin contact, which is a thin (<1 mm), lightweight (˜2.5 ounce) polyester that is breathable, non-shearing, anti-microbial, moisture wicking and quick drying. Examples of appropriate fabrics for this upper layer include the fabric described in U.S. Pat. No. 6,277,770 of Smith, which is incorporated herein by reference. The first layer is cut into a rounded shape, with dimensions in the range of 120-180 mm wide and 105-120 mm high.

The second layer is cut to match the shape of the first layer from a moisture absorbing and retaining breathable fabric that itself has an integrated two-layer structure consisting of an inner, moisture absorbing surface and an outer transporting surface which draws moisture from the moisture absorbing surface, then distributes the moisture across the outer surface for dispersion and more rapid drying without soaking through the outer layer. Such fabrics are commercially available from a number of textile suppliers. The second layer is thin (<1 mm) and lightweight.

The third layer of the assembly, which may be either the same rounded shape as the first two layers or may alternatively be formed as a half rounded shape with its curved edges matching the lower edge of the first layer, is an absorbent, breathable microfiber layer that is flexible and conforms to the body, with a thickness on the order of 1.0 to 3 mm, more preferably less than 2 mm and most preferably less than 1.5 mm. A preferred microfiber material has a density of 180-340 gm/m², which is commercially available from a number of sources. Such microfiber fabrics absorb liquid faster than cotton by virtue of the capillary effect created within tiny spaces between the threads and are able to dry rapidly by communicating the liquid away from the source. The positioning of the third layer provides optimal absorption in the area of the pad that contacts the nipple. The second and third layers may be switched, so that the third layer is sandwiched between the first, skin contact layer and the second layer, or the third layer is sandwiched between the second moisture absorbing, breathable layer and the fourth, outer layer.

The outer layer of the assembly is a thin, lightweight, waterproof, breathable fabric such as the polyester or nylon fabrics with a coating such as polyurethane (PU) or polytetrafluoroethylene (PTFE) for enhancing the waterproof quality. Such fabrics are commercially available from a number of suppliers. In the preferred embodiment, a nylon/LYCRA® blend with a PU finish is used.

A factor that affects the smoothness and comfort of the surface is the assembly method used for combining the multiple layers and finishing the edges of a pad or cushion. The assembly method also effects launderability and drying time. In the preferred embodiment, the multi-layer fabric is stitched using a smooth polyester thread to minimize puckering or bunching, minimize washing and drying time, while maximizing comfort, durability and breathability. Seams, if any, should be free of puckering and are preferably limited to those used for finishing the outer edges of the pad, so that the skin contact surface is substantially seamless. In one embodiment, a friction-enhancing strip, such as silicone or other rubber-like synthetic material, may be formed or attached to selected areas on the outer edges of the pad, away from the nipple contact area, to minimize shifting within the nursing bra cup.

### BRIEF DESCRIPTION OF THE DRAWINGS

FIG. 1 is an exploded perspective view of the four layers of the nursing bra pad according to the present invention.

FIG. 2 is a view of the first inner layer of an embodiment in which pleats are used to form the concave shape to indicate possible pleat locations.

FIG. 3a is a view of the first inner layer of an alternative embodiment formed using a two-step stitching process; FIG. 3b is a side view of the same embodiment.

FIG. 4 is a block diagram showing the steps for assembling the nursing bra pad of the embodiment of FIG. 3.

FIG. 5 is a top view of an embodiment of the outer layer that provides for insertion or removal of additional absorbent material.

### DETAILED DESCRIPTION OF PREFERRED EMBODIMENTS

According to the present invention, a nursing bra pad is provided that is extremely lightweight, has minimal shear for added comfort against the skin, and is easy to launder without degrading comfort or performance.

For purposes of the following description, the skin contact layer, i.e., the inner layer of the nursing bra pad, is described as the “upper layer” or the “first layer”, while the outer layer that is opposite from the skin contact layer is referred to as the “lower layer” or “fourth layer”. These references are provided for convenience with reference to the orientation of the drawings and are not intended to be limiting or to indicate that the layering must always start with either the inner or outer layers during assembly.

In an exemplary embodiment, a multi-layer assembly has a rounded shape, which may be circular or oval (elongated), or a many-sided polygon, such as a hexagon, octagon, decagon, etc., which is effectively rounded. The layers include an upper layer, generally intended for direct skin contact, which is a thin (<1 mm, preferably less than 0.5 mm), lightweight (˜2.5 ounce) polyester that is breathable, non-shearing, moisture wicking and quick drying. Examples of appropriate fabrics for this upper layer include the fabric described in U.S. Pat. No. 6,277,770 of Smith, which is incorporated herein by reference. In the preferred embodiment the fabric is also treated with an anti-microbial compound.

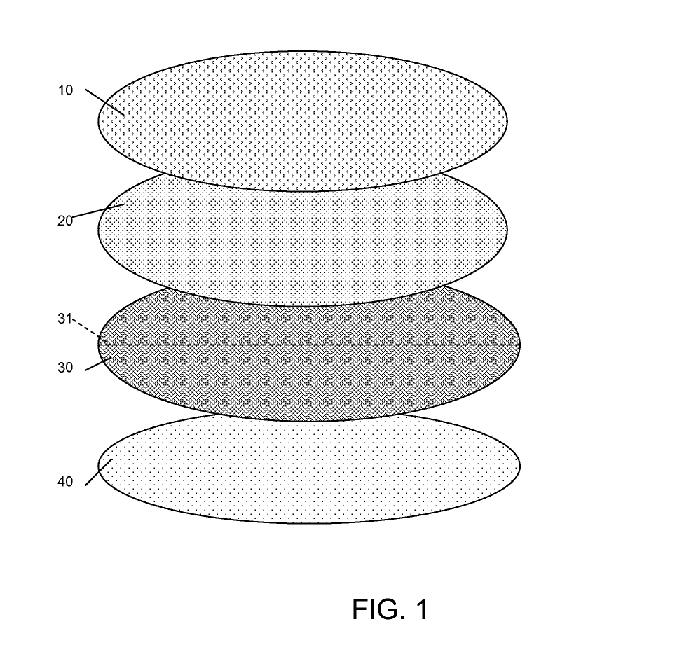

As shown in FIG. 1, the thin, lightweight upper layer 10 should conform well to the user's body and dries quickly. Layer 10 is cut in a rounded shape with an upper portion and a lower portion corresponding to the nipple contact area. The rounded shape has a diameter in the range of 105-180 mm. If an oval or other elongated shape is used, typical dimensions will be around 120-180 mm along the long chord and around 105-120 mm for the short chord. The fabric used for the upper layer has the combined qualities of air porosity (to allow venting through the fabric's planar surface), moisture vapor transport (to enhance comfort), fabric flexibility (also a comfort-enhancing property), extremely small pore size, a non-shearing surface (for reduced friction), and durability to laundering. The fabric thickness is preferably less than 1 mm, with a typical thickness of around 0.2 mm. This combination of properties is provided by weaving fine-denier synthetic filament yarns (polyester or nylon-polyester blend) into a tight plain-weave construction with post finishing processes that maximize fiber coverage and filtration efficiency. Because the fabric is woven from continuous synthetic filament and/or spun yarns, there are no short fibers to irritate skin or become embedded in a wound or sore. The woven filaments are finished to provide a fabric with a mean pore size in the range of 4 to 10 microns, an air permeability of 0.5-30 cubic feet per minute per square foot of fabric at 0.5 inches of water (“cfm,” measured by Federal Test Method Standard (FTM) 5450, also known as ASTM D-737), a mean fabric flexibility in the range of 0.5 to 6.5 grams (bending resistance), and a moisture vapor permeability in excess of 800 g/m² per 24 hours. The coefficient of friction for the fabric is on the order of 0.5 or less, which is approximately one-half of the coefficient of friction of a cotton or polyester/cotton blend with roughly the same fiber and weave.

In the preferred embodiment, a suitable antimicrobial or fluorochemical finish is applied to or incorporated in the fabric. Such finishes are well known in the art. See, e.g., U.S. Pat. No. 4,822,667 of Goad, et al., U.S. Pat. No. 5,069,907 of Mixon, et al., or U.S. Pat. No. 6,762,172 of Elfersy, et al., among many others. An example of a suitable antimicrobial finish that can be applied is a compound of 3-(trimethoxysilyl)-propyl dimethyl octadecyl ammonium chloride (DOW CORNING 5700). The antimicrobial finish protects the fabric against bacteria and fungi, and inhibits the growth of odor-causing bacteria.

As shown in Table 1, compared with materials used in currently commercially-available nursing pads, the preferred skin contact layer (“non-shearing, moisture permeable synthetic”) as described above possesses all of the desired qualities for the user's comfort.

TABLE 1

| | Fabric | | |
|---|---|---|---|
| | Non-shearing, | | |
| | moisture- | | |
| | permeable | Disposable | 100% |
| Feature | synthetic | (Paper) | Cotton |
| Moisture | yes | yes | no |
| wicking | | | |
| Breathable | yes | yes | yes |
| Quick Drying | yes | NA | no |
| (<5 min.) | | | |
| Anti-microbial | yes | NA | no |
| Body | yes | no | no |
| conforming | | | |
| Non-shearing | yes | yes | no |
| Weight | light | light | heavy |
| Thickness | <1 mm | <1 mm | 2 mm |
| Washable | yes | no | yes |
| Recyclable | yes | no | NA |
| potential | | | |

The light, thin top layer is particularly advantageous if quilting or other stitching is used within the center area of the pad, since the thicker the fabric, the greater the chance of puckering and bunching when quilted, which can irritate the mother's nipples.

The second layer 20, also referred to as the first absorbent layer, is cut to match the shape (outline) of the first layer from a moisture absorbing and retaining breathable fabric that itself has an integrated two-layer structure consisting of an inner, moisture absorbing surface and an outer transporting surface which draws moisture from the moisture absorbing surface, then distributes the moisture across the outer surface for dispersion and more rapid drying without soaking through the outer layer. One process for forming such an engineered fabric involves a combination of hydrophobic and hydrophilic yarns, which may be effectively integrated by weaving, plate knitting or other techniques known within the textile industry. These moisture management fabrics act to prevent, or minimize, the collection of moisture against the body and in the fabric layer that is next to the wearer's skin. The moisture, in liquid or vapor form, leaves the skin surface and diffuses, or wicks, through the hydrophobic fibers to be absorbed by the hydrophilic fibers in the outer fabric layer. An example of such a fabric is described in U.S. Patent Publication No. 2003/0182922 of Peters, which is incorporated herein by reference. Fabrics with these characteristics are commercially available from a number of textile suppliers. The second layer should be thin (<1 mm) and lightweight.

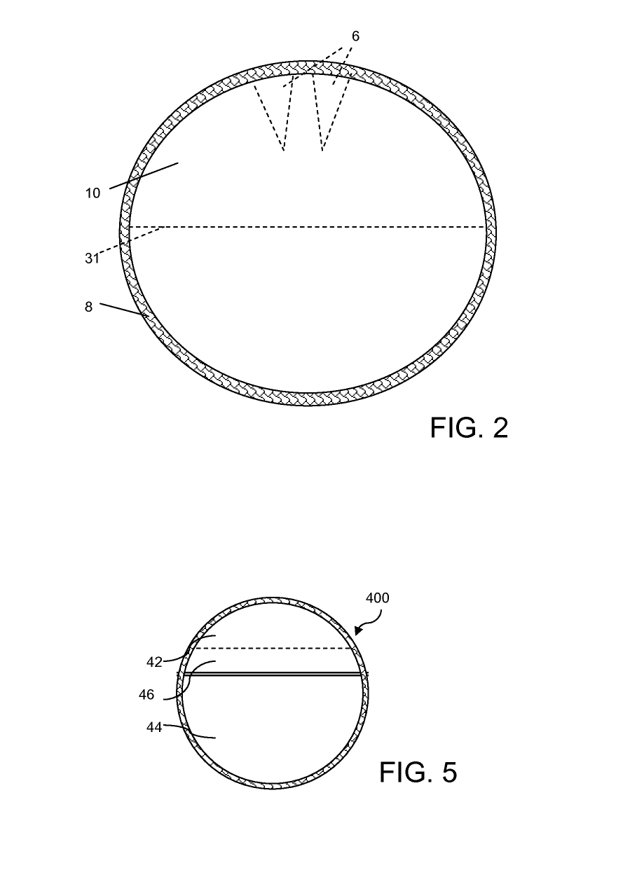

The third layer 30 of the assembly, also referred to as the second absorbent layer, which is used to enhance the moisture absorbency, is an absorbent, breathable polyester microfiber that is flexible and conforms to the body, with a thickness on the order of 1.0 to 3 mm, more preferably less than 2 mm and most preferably less than 1.5 mm. The shape of the third layer should match that of the first and second layers, or alternatively, may be semi-rounded, e.g., a half-circle, as indicated by dashed line 31 in FIGS. 1 and 2, to provide extra absorbency at the lower portion of the pad corresponding to the nipple contact area. A preferred microfiber for the third layer has a density of 180-340 gm/m². Such microfiber materials are available from a number of commercial suppliers. One example of an appropriate microfiber material is described in U.S. Pat. No. 6,381,994 of Lee, which is incorporated herein by reference. Such microfiber fabrics are formed by weaving synthetic filaments or fibers that have many channels extending along the fiber lengths, thus creating fine voids within their cross-sections. Microfiber fabrics of this construction are able to absorb liquid faster and in greater volume than cotton due to the capillary effect produced by the voids and channels within the fibers and, thus, are able to dry rapidly by communicating the liquid away from the source.

Table 2 provides a comparison of relevant characteristics of the absorbent third layer of the multi-layer fabric of the preferred embodiment of the present invention to the materials used in currently commercially-available products.

TABLE 2

| | Fabric | | |
|---|---|---|---|
| | Absorbent | Disposable | |
| Feature | microfiber | (paper) | polyester |
| Absorbs fluids | yes | yes | no |
| Washable | yes | no | yes |
| Body- | yes | no | no |
| conforming | | | |
| Breathable | yes | no | no |
| Discourages | yes | NA | NA |
| microbes | | | |
| Thickness | 2 mm | | 3-4 mm |
| Recyclable | yes | no | NA |
| potential | | | |

The third layer (second absorbent layer) may be omitted entirely in cases where the woman is experiencing only light milk leakage. Customization of the absorbency can be provided by including a closable opening in the outer layer to allow additional absorbent material to be interested. As illustrated in FIG. 5, the modified outer layer 400 is formed from upper and lower semi-circles 42 and 44, respectively, which are sewn around their edges (during the stitching step) but left open across the body of the pad where they overlap to allow the flap 46 to be opened to insert or remove additional absorbent material such as the microfiber material used in the third layer. A similar modification in the skin contact layer can be made to permit insertion of a topical wound dressing for treatment of a breast or nipple infection. In this variation, the overlapping area would need to be shifted as much as possible toward the top of the pad so that the fabric edges would not come into contact with the already irritated nipple.

The outer layer of the assembly is a thin, lightweight, waterproof, breathable synthetic fabric such as the polyester or nylon coated with polyurethane (PU) or polytetrafluoroethylene (PTFE). Such fabrics are available from a number of different textile manufacturers worldwide.

TABLE 3

| | Fabric | | |
|---|---|---|---|
| | Breathable, | | |
| | waterproof | | |
| Feature | synthetic | Disposable | polyester |
| Waterproof | yes | yes | yes |
| Breathable | yes | yes | no |
| Body- | yes | no | no |
| conforming | | | |
| Washable | yes | no | yes |
| Weight | light | light | heavy |
| Thickness | <1 mm | <1 mm | 2-3 mm |
| Recyclable | yes | no | NA |
| potential | | | |

Exemplary characteristics of a preferred fabric for the outer (bottom) layer of the inventive multi-layer support surface include a fabric weight on the order of 80-100 gm/m² that is waterproof and breathable. The breathability of the outer layer is measured according to moisture vapor transfer rate (MVTR), expressed in units of grams/meter²/day. For purposes of the present invention, a MVTR range of 5,000 to 20,000 g/m²/day is preferred. Table 4 below provides a comparison of the breathability of three different fabrics after applying 2 ml of water to the fabric. The degree of dryness was tested on the underside of the fabric, which would correspond to the moisture to which a mattress beneath the fabric would be exposed. The MVTR values were provided by exemplary manufacturers and were not independently confirmed.

TABLE 4

| | Fabric (MVTR) | | |
|---|---|---|---|
| | Fabric #1 | Fabric #2 | Fabric #3 |
| Hr:Min after | (7,000 g/ | (7,000 g/ | (10,000 g/ |
| applying water | m²/day) | m²/day) | m²/day) |
| 0:00 | 80% dry | 80% dry | 80% dry |
| 0:05 | 85% dry | 85% dry | 85% dry |
| 0:10 | 90% dry | 90% dry | 90% dry |
| 0:15 | 95% dry | 95% dry | 95% dry |
| 0:45 | 98% dry | 98% dry | 98% dry |
| 2:30 | Completely | Completely | Completely |
| | Dry | Dry | Dry |

The tests indicated that MVTR ratings of 7,000 and 10,000 g/m²/day were equally effective in drawing away the moisture at a desired rate.

In the preferred embodiment, the fourth, outer layer is a flexible, soft nylon66 (89%)/LYCRA® (11%) blend with a PU finish to enhance the waterproof characteristics of the fabric. Alternatively, a TEFLON® finish may be used. The fabric may have multi-directional stretchability, ideally with a 4-way stretch, e.g., SPANDEX®.

A factor that affects the smoothness and comfort of the surface is the assembly method used for combining the multiple layers and finishing the edges of a pad. The assembly method and materials also influence launderability and drying time. For example, a cotton thread tends to have a heavier weight, less give (stretchability) and a longer drying time. In the preferred embodiment, the multi-layer fabric is stitched using a smooth polyester thread. The polyester thread is stronger, lighter, and faster drying with better give compared to cotton or cotton-blends. All layers of the multi-layer assembly are simultaneously stitched around their rounded edges with polyester thread as illustrated in FIG. 2 to define seam 8. In one embodiment, at the same time that seam 8 is created, pleats may be formed along the upper portion of the pad by folding over the fabric along the dashed lines 6 to form a concave, cup-like shape to conform to the shape of the breast. It may be possible to create a quilted pattern within the central area of the pad, but stitching should preferably be limited to the upper portion, away from the nipple contact area in the lower portion, to avoid irritation by the thread edges.

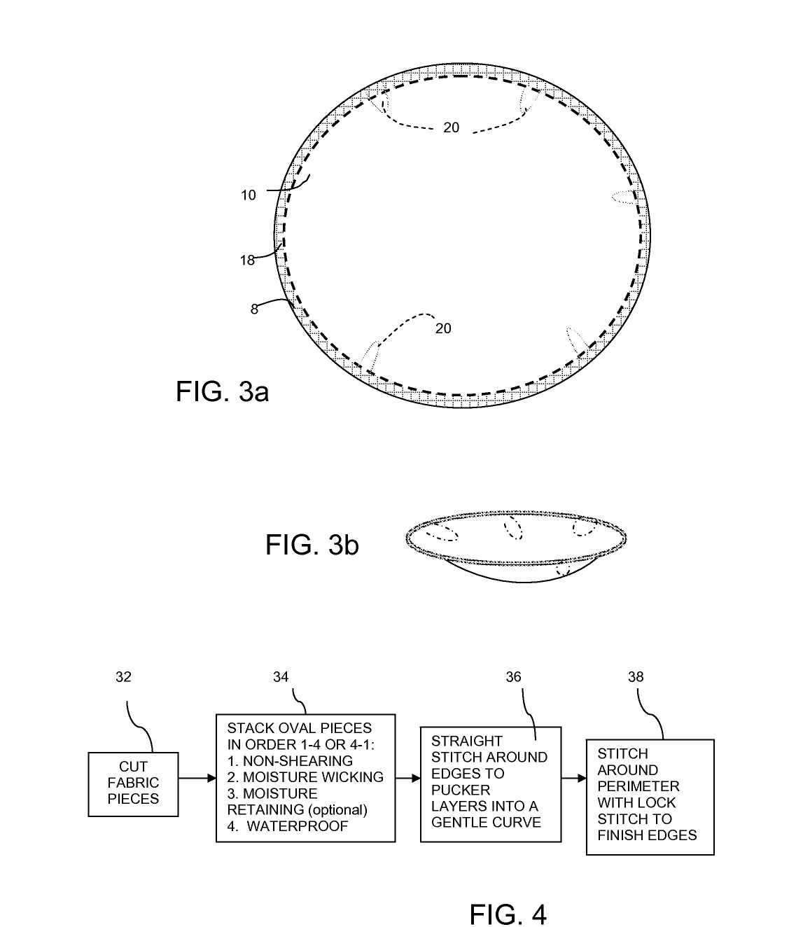

A preferred embodiment of the multi-layer assembly is shown in FIGS. 3a and 3b. In this embodiment, the pleats or darts defined by folding over the fabric, as shown in FIG. 2, are replaced by a two-step stitching process. In the first step of the process, polyester thread is used to create a straight stitch 18 around the entire periphery of the rounded shape. The tension on the sewing machine is adjusted to produce a puckering around the edges of the assembly, drawing the edges of the layers in to create many tiny puckers 20. (The puckers shown in the figures are exaggerated in size for illustration and appear larger than would actually be created using the described technique.) The puckers are very small (less than 10 mm, preferably around 1-3 mm) and numerous, randomly distributed around the edges, thus conveying a gentle curvature to the assembly, as illustrated in FIG. 3b. Because the puckers 20 are small and generally confined to the outer periphery, there is little risk of creating an uneven surface at the center of the pad that might irritate the user's nipple. After the first stitching is completed, the edges of the pad are finished using a lock stitch 8.

FIG. 4 provides a block diagram of the steps of the process for assembling the nursing bra pad of FIGS. 3a and 3b. In step 32, rounded pieces are cut from each of the four fabrics described above, then stacked on top of each other from inner layer to outer layer or vice versa (step 34). In the first stitching step 36, a straight stitch is made around the edges of the layers to cause the assembly to pucker, forming a concave shape with a gentle curve. In the second stitching step 38, a lock stitch is used to secure the edges of the assembly.

In an alternative embodiment, the stitching may be replaced with an appropriate adhesive, thermal bonding, welding or other means known for attaching fabric layers, with caution to avoid altering the softness, pliability and other desirable characteristics of the fabric layers. Both the puckering and finishing steps should be achievable using alternative fastening techniques that are known to those in the art.

Generally, the combination of the cup-like shape and pleats or puckers should be sufficient to hold the pad within the bra cup to prevent shifting. However, in one embodiment, a friction-enhancing surface such as rubber or silicone may be formed on a portion of the outer layer to assist in holding the pad in position within the nursing bra. If used, the friction-enhancing surface should only cover a small area of the total pad area so that breathability is not impacted. Further, the surface would preferably be located only at the upper portion of the pad so that conformability and comfort are not affected in the areas nearest the nipple contact area.

The total combined thickness of the multi-layer assembly of the present invention is on the order of 2-5 mm, preferably at the lower end of the range. The minimal thickness enhances the assembly's pliability and its ability to conform comfortably to the user's breast without adding bulk. It also provides a pad that is easy to launder and dries quickly. The inventive pads provide excellent absorption of liquids while effectively drawing the moisture away from the skin to avoid skin irritation and bacterial or fungal growth. Further, the outer waterproof layer prevents leaking of milk that can soil clothing.

While the preferred embodiments of the invention have been described, modifications can be made and other embodiments may be devised without departing from the spirit of the invention and the scope of the appended claims.

1. A nursing bra pad comprising:
a skin contact layer comprising a woven fabric of synthetic filament yarns woven to provide air permeability and moisture vapor permeability and having a thickness of less than 1 mm, the skin contact layer having a generally rounded shape;
a first absorbent layer comprising a synthetic breathable fabric having a moisture-absorbing inner surface and a moisture-transporting outer surface for drawing moisture away from the skin contact layer and transporting the moisture to the outer surface of the first absorbent layer, wherein the first absorbent layer has a rounded shape to match the rounded shape of the skin contact layer;
an outer layer having a shape to match the rounded shape of the skin contact layer, disposed opposite the skin contact layer so that the first absorbent layer is sandwiched therebetween, the outer layer comprising a breathable, waterproof synthetic fabric having a thickness of less than 1 mm;
a seam formed around the rounded edges of each of the skin contact layer, the first and second absorbent layers and the outer layer to attach the layers; and
stitched means for forming a concave shape with the combined layers.

2. The nursing bra pad of claim 1, further comprising a second absorbent layer sandwiched between the skin contact layer and the outer layer, the second absorbent layer comprising a breathable, moisture absorbing and retaining microfiber fabric having a shape to match at least a portion of the rounded shape of the skin contact layer.

3. The nursing bra pad of claim 1, wherein the stitched means comprises a straight stitch that creates puckers near the edges of the layers.

4. The nursing bra pad of claim 1, wherein the skin contact layer is treated with an anti-microbial finish.

5. The nursing bra pad of claim 1, wherein the skin contact layer has a mean pore size of 4 to 10 microns.

6. The nursing bra pad of claim 1, wherein the skin contact layer has a coefficient of friction of 0.5 or less.

7. The nursing bra pad of claim 1, wherein the skin contact layer has a thickness of around 0.2 mm.

8. The nursing bra pad of claim 1, wherein the skin contact layer is woven from a continuous fine-denier filament yarn of polyester or polyester blend.

9. The nursing bra pad of claim 1, wherein the first absorbent layer comprises an integrated combination of hydrophilic and hydrophobic yarns.

10. The nursing bra pad of claim 1, wherein the first absorbent layer has a thickness of less than or equal to 1 mm.

11. The nursing bra pad of claim 2, wherein the microfiber fabric of the second absorbent layer has a density of 180-340 gm/m².

12. The nursing bra pad of claim 1, wherein the outer layer has a moisture vapor transfer rate of 5,000 to 20,000 g/m²/day.

13. The nursing bra pad of claim 1, wherein the outer layer has a finish of polyurethane.

14. The nursing bra pad of claim 1, wherein the outer layer is a nylon-LYCRA® blend.

15. The nursing bra pad of claim 1, wherein the outer layer comprises two partially overlapping semi-circles that define a flap for insertion or removal of additional absorbent material.

16. A nursing bra pad comprising:
a layered assembly of rounded fabric sheets having dimensions to fit within a nursing bra cup and a total thickness of less than 5 mm, comprising:
an inner layer for contacting a user's skin, the first layer comprising a woven fabric of synthetic filament yarns woven to provide air permeability and moisture vapor permeability and having a thickness of less than 1 mm;
an absorbent layer comprising a synthetic breathable fabric having a moisture-absorbing inner surface and a moisture-transporting outer surface for drawing moisture away from the skin contact layer and transporting the moisture to the outer surface of the second layer;
an outer layer comprising a breathable, waterproof synthetic fabric having a thickness of less than 1 mm; and
at least one stitched means for attaching the edges of the layered fabric sheets, for puckering the layers near the edges to form a concave shape within the layers and finishing the edges of the layered fabric sheets.

17. The nursing bra pad of claim 16, further comprising a second absorbent layer disposed between the inner layer and the outer layer, the second absorbent layer comprising a breathable, moisture absorbing and retaining microfiber fabric.

18. The nursing bra pad of claim 16, wherein the inner layer is treated with an anti-microbial finish.

19. The nursing bra pad of claim 16, wherein the inner layer has a mean pore size of 4 to 10 microns.

20. The nursing bra pad of claim 16, wherein the inner layer has a coefficient of friction of 0.5 or less.

21. The nursing bra pad of claim 16, wherein the inner layer has a thickness of around 0.2 mm.

22. The nursing bra pad of claim 16, wherein the inner layer is woven from a continuous fine-denier filament yarn of polyester or polyester blend.

23. The nursing bra pad of claim 16, wherein the absorbent layer comprises an integrated combination of hydrophilic and hydrophobic yarns.

24. The nursing bra pad of claim 16, wherein the absorbent layer has a thickness of less than or equal to 1 mm.

25. The nursing bra pad of claim 17, wherein the microfiber fabric of the second absorbent layer has a density of 180-340 gm/m².

26. The nursing bra pad of claim 16, wherein the outer layer has a moisture vapor transfer rate of 5,000 to 20,000 g/m²/day.

27. The nursing bra pad of claim 16, wherein the outer layer has a finish of polyurethane.

28. The nursing bra pad of claim 16, wherein the outer layer is a nylon-LYCRA® blend.

29. The nursing bra pad of claim 16, wherein the at least one stitched means comprises a first straight stitch having a tension adjusted to pucker the fabric layers and a second lock stitch.

30. The nursing bra pad of claim 16, wherein the outer layer comprises two partially overlapping semi-circles that define an openable flap for insertion or removal of additional absorbent material.

31. A nursing bra pad comprising:
a layered assembly of rounded fabric sheets having dimensions to fit within a nursing bra cup and a total thickness of less than 5 mm, comprising:
a first layer for contacting a user's skin, the first layer comprising a woven fabric of synthetic filament yarns woven to provide air permeability and moisture vapor permeability and having a thickness of less than 1 mm;
a second layer comprising a synthetic breathable fabric having a moisture-absorbing inner surface and a moisture-transporting outer surface for drawing moisture away from the skin contact layer and transporting the moisture to the outer surface of the second layer;
a third layer comprising a breathable, moisture absorbing and retaining microfiber fabric;
a fourth layer comprising a breathable, waterproof synthetic fabric having a thickness of less than 1 mm; and
means for attaching the edges of the layered fabric sheets, for puckering the layers near the edges to form a cup-like shape within the layers and finishing the edges of the layered fabric sheets.

32. The nursing bra pad of claim 31, wherein the first layer is treated with an anti-microbial finish.

33. The nursing bra pad of claim 31, wherein the outer layer has a finish of polyurethane.

34. The nursing bra pad of claim 31, wherein the outer layer comprises two partially overlapping semi-circles that define a flap for insertion or removal of additional absorbent material.
